(19) United States
(12) Patent Application Publication
(10) Pub. No.: US 2021/0145643 A1
(43) Pub. Date: May 20, 2021

(54) FRAME FOR EYEWEAR PROVIDING IMPROVED PROTECTION AGAINST IONISING RADIATION AND RADIOPROTECTIVE EYEWEAR COMPRISING SUCH A FRAME

(71) Applicant: ORANO CYCLE, Châtillon (FR)

(72) Inventor: Jacky Jehanno, Gaujac (FR)

(21) Appl. No.: 17/047,507

(22) PCT Filed: Apr. 12, 2019
(86) PCT No.: PCT/FR2019/050868

§ 371 Date: Oct. 14, 2020

(30) Foreign Application Priority Data
Apr. 16, 2018 (FR) 1853320

(51) Int. Cl. A61F 9/04; A61F 9/02; G21F 3/02; G02C 3/00; G02C 11/00; G02C 5/00; G02C 7/02

(52) U.S. Cl. CPC A61F 9/045; A61F 9/027; G21F 3/02; G02C 2200/02; G02C 11/12; G02C 5/001; G02C 7/02; G02C 3/003

(57) ABSTRACT

A frame for eyewear for protecting against ionizing radiation, comprising a frontal portion extending laterally by the two lateral protective elements, and a device for maintaining the frame on the face of a user under the conditions under which it is worn, and characterized in that the lateral protective elements are integrally formed with the frontal portion and in that the frontal portion and the lateral protective elements are made of a radio-attenuation material. Further, eyewear for protecting against ionizing radiation comprising such a frame and to an assembly comprising this eyewear and a device for distributing weight.

### TECHNICAL FIELD

The invention relates to the field of individual protection against ionising radiation and, in particular, against X or gamma type electromagnetic radiation.

More specifically, it relates to an eyewear frame providing an improved protection against ionising radiation as well as protective eyewear against ionising radiation comprising such a frame.

This eyewear will be more simply referred to as “radiation protective eyewear” in what follows.

The invention also relates to an assembly comprising radiation protective eyewear and a weight take-up device for this eyewear.

The invention finds application in all the fields in which persons are likely to be exposed to ionising radiation while practicing their professional activity and, especially, in the fields of medicine (diagnostic radiology, interventional radiology, radiation therapy, brachytherapy, nuclear medicine, etc), basic and applied research, and of nuclear industry (mining activities, uranium conversion, treatment of used nuclear fuels, manufacture of new nuclear fuels, etc.).

### State of Prior Art

Council Directive 2013/59/Euratom of 5 Dec. 2013 laying down the basic standards for health protection against dangers arising from exposure to ionising radiation lowered the equivalent dose limit to the crystalline lens for workers. It now requires, in planned situations of exposure related to the practice of a professional activity, an equivalent dose limit to the crystalline lens of 20 millisieverts (mSv) per year, on average over defined periods of 5 years, without exceeding 50 mSv over a single year, whereas this limit was 150 mSv per year until then.

Complying with this Directive necessitates a suitable dosimetry monitoring of workers and/or systematic wearing personal eye radiation protective equipment which not only have to efficiently protect the crystalline lens but also meet ergonomic criteria for workers to easily submit to the obligation of wearing this equipment.

There are three types of individual eye radiation protective equipment: eyewear with or without prescription glasses, coverall eyewear which is for persons wearing prescription eyewear and peaks (also called “peaked caps”) which can be worn both with or without prescription eyewear and which is specially dedicated to the medical field since it also provides protection against spatters of biological liquids such as blood.

Among these three types of equipment, eyewear is the simplest to use by far and likely to be worn most easily, frequently, or even systematically, by workers.

Radiation protective eyewear which is presently commercially available is designed to provide either a solely frontal protection through the presence of lead glasses or a frontal protection plus a side protection, in which case this eyewear comprises, in addition to lead glasses, side protective elements with a limited size (typically in the order of 2 cm high by 1.5 cm wide) which are assembled to side rims comprised by the frame or inserted into these side rims.

In both cases, the frame is of a plastic material transparent to ionising radiation.

Frontal or side protective eyewear efficiently attenuates ionising radiation impinging on the lead glasses and side protective elements.

However, it attenuates neither ionising radiation impinging on the frame nor those coming from the interaction of ionising radiation with skin zones covered by the frame or surrounding this frame. But, according to their direction of propagation, these ionising radiation, which are not attenuated by eyewear, are also likely to reach the crystalline lens.

Therefore, the purpose set by the inventor is to provide radiation protective eyewear offering an improved protection of the crystalline lens against ionising radiation.

One purpose is also that this eyewear additionally is ergonomic and, in particular, comfortable to wear as well as aesthetic in order to promote its systematic use by workers which are subjected to an exposure to ionising radiation.

Another purpose is that the production cost of this eyewear enables it to be widely distributed in professional sectors in which an individual eye protection against ionising radiation is needed.

### DISCLOSURE OF THE INVENTION

These purposes are achieved by the invention which first provides a frame for protective eyewear against ionising radiation, which comprises a frontal part, or face, being laterally extended by two side protective elements, as well as a device for holding the frame on a user's face in wearing condition, and which is characterised in that the side protective elements are as a single piece with the frontal part and in that the frontal part and the side protective elements are made of a radiation attenuating material comprising a polymer matrix in which a radiation attenuating load is dispersed. In what precedes and what follows:
by “radiation attenuating load”, it is meant any material which is in a divided form, preferably as particles, and which is capable of interacting with photons from an ionising radiation and absorbing part of the energy of these photons; and
by “radiation attenuating material”, it is meant a material which, since it comprises a radiation attenuating load, is also capable of interacting with photons from an ionising radiation and absorbing part of the energy of these photons.

The ionising radiation can especially be an X type electromagnetic radiation if this is produced by an X-ray generator within which a potential difference conventionally ranging from several tens to several hundreds of kilovolts (kV) is applied, or a gamma type electromagnetic radiation if this is emitted by one or more radioactive atoms upon disintegrating.

Furthermore, by “polymer”, it is meant a homopolymer, that is a polymer from polymerising a single species of monomers, as well as a copolymer, that is a polymer from copolymerising several species of monomers.

In accordance with the invention, the radiation attenuating load preferably comprises particles of at least one metal selected from lead, rare earths, bismuth, antimony, tin, tungsten, barium, tantalum, or of at least one compound selected from alloys and oxides of these metals.

It is reminded that rare earths are a group of metals comprising scandium (Sc), yttrium (Y) and all the lanthanides, the latter corresponding to the 15 chemical elements listed in the Mendeleev periodic table of elements from atomic number 57 for lanthanum (La) to atomic number 71 for lutetium (Lu).

Thus, the attenuating load can especially—or consists of—particles of:
lead or lead oxide and, in particular, lead monoxide of the formula PbO;
erbium or erbium oxide and, in particular, erbium sesquioxide (III) of the formula Er₂O₃;
praseodymium or praseodymium oxide and, in particular, praseodymium oxide(III-IV) of the formula Pr₆O₁₁;
bismuth or bismuth oxide such as bismuth sesquioxide of the formula Bi₂O₃;
a mixture of erbium oxide and praseodymium oxide such as a mixture of Er₂O₃ and Pr₆O₁₁, for example in mass proportions of 55% to 65% of Er₂O₃ and 35% to 45% of Pr₆O₁₁, with respect to the mass of the mixture;
a mixture of erbium oxide, praseodymium oxide and bismuth oxide such as a mixture of Er₂O₃, Pr₆O₁₁ and Bi₂O₃, for example in mass proportions of 30% to 45% Er₂O₃, 20% to 30% of Pr₆O₁₁ and 30% to 45% of Bi₂O₃, with respect to the mass of the mixture; or even
a mixture of bismuth oxide, tungsten oxide and lanthanum oxide such a mixture of Bi₂O₃, tungsten trioxide of the formula WO₃ and lanthanum trioxide of the formula La₂O₃, for example in mass proportions of 70% to 90% by mass of Bi₂O₃, 5% to 15% by mass of WO₃ and 5% to 15% of La₂O₃, with respect to the mass of the mixture.

The radiation attenuating load is preferentially selected depending on the photon energy of ionising radiation to be attenuated by the radiation protective eyewear and, therefore, by its frame.

Thus, in facilities for manufacturing MOX (Mixed OXide) type nuclear fuel implementing powders of uranium and plutonium oxides, major radiation being X radiation from radio-isotopes of plutonium especially, americium and uranium 237 and corresponding to ionising radiation with a photon energy of 60 keV and 208 keV respectively, a bismuth- or bismuth-oxide based radiation attenuating load will be for example selected.

The polymer of the matrix is selected depending on the qualities desired to be imparted to the frame, in particular in terms of compliance, flexibility, weight, solidity (impact resistance, chemical resistance, etc) and aesthetics (colours, textures, etc), as well as the production cost of this frame.

Thus, it can especially be a cellulose acetate such as those marketed by MAZZUCHELLI and PLASTIMOD, a cellulose acetopropionate such as those marketed by EASTMAN Chemical Company under the Tenite™ references, a polyamide or copolyamide such as those marketed by EMS-Grivory under the Grilamid™ references or those marketed by ARKEMA under the Rielsan™ references, an epoxide resin such as Optyl™, or a polyurethane such as those marketed by COVESTRO under the Desmopan™ Reference.

Among these, preference is given to:
on the one hand, polyamides and copolyamides and, especially, Grilamid™ TR 90 from EMS-Evory due to its extreme lightness (which is a highly appreciable quality for making wrap-around frames), its resilience (since it is a shape memory polymer) and its exceptional resistance, in particular impact resistance, properties and
on the other hand, polyurethanes.

In accordance with the invention, the mass proportion of the radiation attenuating load in the radiation attenuating material and/or the thickness of this material are preferentially selected so that this material has a lead equivalence at least equal to 0.4 mm, that is it ensures protection against ionising radiation equivalent to that imparted by a 0.4 mm thick lead plate.

This mass proportion, which varies depending on the composition of the radiation attenuating load used, the thickness of the radiation attenuating material as well as the attenuation level of ionising radiation needed and which is therefore to be adapted on a case by case basis—which those skilled in the art will be able to do—is typically between 20% and 95% with respect to the mass of the radiation attenuating material.

The frame of the invention can have very different shapes depending on the use intended for the radiation protective eyewear and, especially, the protective surface needed within the scope of this use but also depending on ergonomic and aesthetic criteria this eyewear is desired to meet.

Firstly, it can be a frame the frontal part of which is configured to embed two optical glasses each of which are for covering one of both orbital areas of a user in wearing condition.

Alternatively, it can be a so-called “mono-shield” frame, that is the frontal part of which is configured to embed only one optical glass which is for covering both orbital areas of a user in wearing condition.

In either case, the frame can have a more or less covering shape, or even wrap-around shape according to the protective surface needed.

Thus, it can be a frame the frontal part of which is planar or with a low degree of curvature, that is with a bending angle between 0° and 10°.

Alternatively, it can be a frame the frontal part of which has a strong degree of curvature to fit the shape of the face more closely, that is with a bending angle greater than 10° and up to 30°.

Furthermore, it can be a frame in which only the frontal part of this frame and the side protective elements take part in ensuring radiation protection.

Alternatively, it can be a frame which additionally comprises two supra-orbital protective elements and/or two infra-orbital protective elements, these supra-orbital and/or infra-orbital protective elements being as a single piece with the frontal part and the side protective elements.

The device for holding the frame on a user's face in wearing condition can comprise two arms hinged to the side protective elements, in which case these arms can be either of a radiation attenuating material as the rest of the frame or consist of radiation attenuating load-free polymer, this polymer can be the same as or different from that forming the matrix of the radiation attenuating material.

For ergonomic or aesthetic reasons, the arms can also be “bi-material” arms, that is consisting partly of a first material and partly of a second material having different properties, for example with flexibility, weight, solidity or appearance, from those of the first material.

Furthermore, the arms can be rectilinear with however a curvature of their ends to ensure comfort and stability to a user, or curved so as to snugly fit a user's head. In all cases, their length can be adjustable.

Alternatively, the device for holding the frame on a user's face in wearing condition can comprise a headband detachably or not detachably connected to the side protective elements, in which case this headband is advantageously of an elastic material, for example an elastic textile or elastomer such as neoprene, and adjustable to the circumference of a user's head.

In accordance with the invention, the frontal part of the frame preferably has a height between 3.5 cm and 5.5 cm and a width between 13 cm and 17 cm.

As for the side protective elements, they preferably have a length at least equal to 2 cm and, even better, between 2.5 cm and 3.5 cm, and a height which, at any point in their length, is at least equal to 80% of the height of the frontal part of the frame at junction zones between this frontal part and the side protective elements.

As is usual in the field of eyewear frames, the frame advantageously additionally comprises a bearing element on a user's nose in wearing condition, which is preferably a saddle bridge. This saddle bridge can be either as a single piece with the frontal part of the frame, in which case it is made of the same radiation attenuating material as that forming this frontal part and the side protective elements of the frame, or assembled to the frontal part of the frame, in which case it is preferentially of a flexible elastomer material such as a silicone for more comfort, wherein this compliant elastomer material can comprise a radiation attenuating load, preferably identical to that present in the frontal part and the side protective elements of the frame.

One object of the invention is also protective eyewear against ionising radiation, which comprises one or two radiation attenuating optical glasses embedded in a frame and which is characterised in that the frame is as previously defined.

In accordance with the invention, the radiation attenuating optical glass(es) can be glasses which do not ensure any optical correction or, on the contrary, glasses which correct an optical defect such as myopia, hypermetropia, astigmatism alone or associated with myopia or hypermetropia, or presbiopia alone or associated with a myopia, hypermetropia and/or astigmatism, in which case they can be unifocal, bifocal or progressive.

Preferably, this/these glass(es) are lead glasses, in particular lead-loaded mineral glasses, typically as oxide(s). They can have a lead equivalence of 0.50 mm, or even 0.75 mm. Such glasses are especially available from SCHOTT company and HOYA company.

The presence of a radiation attenuating load in the frontal part and side rims of the radiation protective eyewear frame of the invention, or even in the arms of this eyewear if it includes arms, leads to an increase in the weight of the eyewear relative to what this weight would be if the frame was free of radiation attenuating load.

For this reason, in order that this increase in weight does not result in an ergonomic loss, it is provided according to the invention to optionally provide the eyewear frame with a first attaching element which is configured to attach to a second attaching element comprised by a weight take-up device of the eyewear when the eyewear and weight take-up device are worn by a user, the eyewear being worn on the user's face and the weight take-up device being worn on the user's skull.

In accordance with the invention, the first attaching element is advantageously a magnet which is assembled to the frame or integrated into this frame.

Another object of the invention is an assembly comprising radiation protective eyewear such as previously defined, and a weight take-up device, in which the eyewear comprises a first attaching element, the weight take-up device comprises a second attaching element and in which the first and second attaching elements are configured to attach to each other when the eyewear and weight take-up device are worn by a user, the eyewear being worn on the user's face and the weight take-up device being worn on the user's skull.

In accordance with the invention, the weight take-up device can be of the headgear type, that is configured to cover the top of the user's skull in wearing condition, in which case it can be a cap, a knit cap or similar, or of the hair band type, that is configured to surround only the frontal part of the skull.

Preferably, the first and second attaching elements are magnets with opposite polarity.

The invention has numerous advantages.

Indeed, as shown in FIGS. 6A, 6B, 7, 8A and 8B, the radiation protective eyewear of the invention offers, by its frame, a protective surface against ionising radiation which is dramatically increased relative to that provided by the radiation protective eyewear of the state of the art with, as a bonus, a better angular protection in the median and paramedian vertical planes of the crystalline lens as well as in the median and paramedian horizontal planes of the crystalline lens.

The lead equivalent of the present glasses of radiation protective eyewear of the state of the art which is typically of 0.75 mm was selected to comply with a given dosimetry level, which arises from energy deposited by direct and indirect ionising radiation on the crystalline lens. Since the radiation protective eyewear of the invention provides an increased protective surface area with an increasing number of attenuated direct radiation and a decreasing contribution of indirect radiation, it makes it possible to contemplate a reduction in the lead equivalent necessary for glasses, and therefore, a decrease in the weight of these glasses and, thus, in the weight of the whole eyewear, with an equivalent dosimetry level.

Furthermore, the radiation protective eyewear of the invention offers an improved protection relative to that provided by the radiation protective eyewear of the state of the art without sacrificing ergonomics and aesthetics suitable for promoting their systematic use by workers which are subjected to exposure to ionising radiation.

Due to its advantages, the eyewear of the invention is particularly adapted to be used in nuclear industry, especially for handling powders of MOX type nuclear fuels.

However, it is also possible to use this eyewear in all the other professional sectors in which protection against ionising radiation is needed and, in particular, in the medical sector for diagnostic radiology, interventional radiology, radiation therapy, brachytherapy, nuclear medicine, etc.

Further characteristics and advantages of the invention will appear from the following additional description.

It is obvious that this additional description is only given by way of illustrating the object of the invention and should in no way be construed as limiting this object.

### BRIEF DESCRIPTION OF THE FIGURES

FIGS. 1A and 1B illustrate two examples of radiation protective eyewear of the state of the art, with frontal and side protections.

FIGS. 2A and 2B schematically and partially represent a person's head, in a left side view, wearing radiation protective eyewear such as illustrated in FIG. 1A.

FIG. 3 schematically and partially represents the person's head, in a front view, wearing radiation protective eyewear such as illustrated in FIG. 1A.

FIGS. 4A and 4B schematically and partially represent a person's head, in a top view, wearing radiation protective eyewear such as illustrated in FIG. 1A.

FIGS. 5A, 5B, 5C and 5D illustrate four examples of radiation protective eyewear of the invention.

FIGS. 6A and 6B are similar representations to those of FIGS. 2A and 2B but for a person wearing radiation protective eyewear such as illustrated in FIG. 5A.

FIG. 7 is a similar representation to that of FIG. 3 but for a person wearing radiation protective eyewear such as illustrated in FIG. 5A.

FIGS. 8A and 8B are similar representations to those of FIGS. 4A and 4B but for a person wearing radiation protective eyewear such as illustrated in FIG. 5A.

FIG. 9A schematically represents a person's head, in a front view, wearing an example of radiation protective eyewear/weight take-up device assembly of the invention, whereas FIG. 9B schematically and partially represents this person's head but in a left side view.

### DETAILED DISCLOSURE OF PARTICULAR EMBODIMENTS

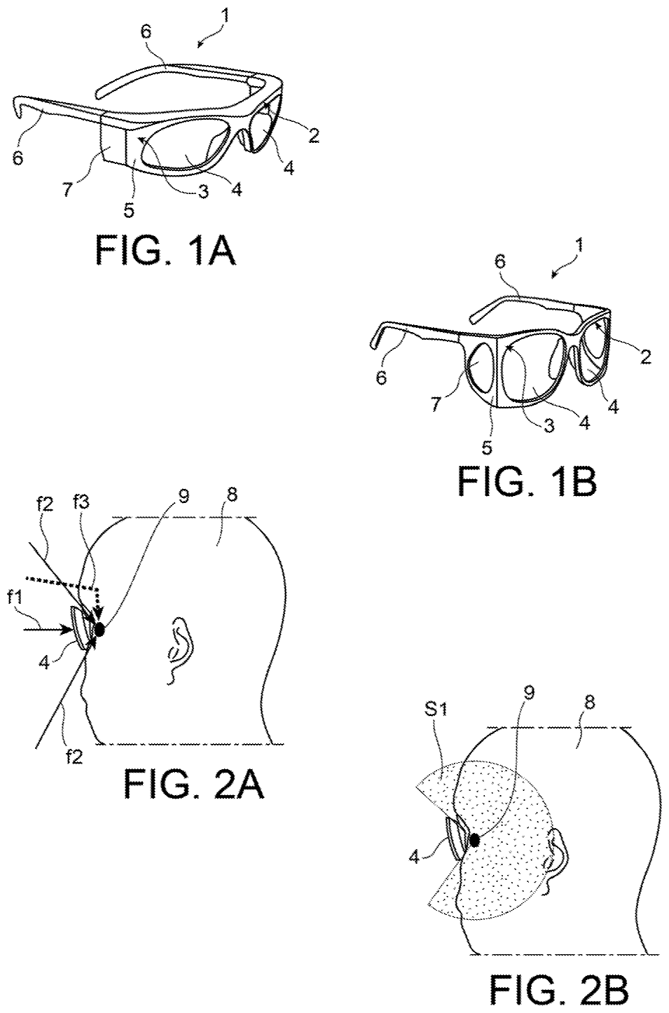

First, FIGS. 1A and 1B are referred to, which illustrate two examples of radiation protective eyewear of the state of the art, with frontal and side protections.

This eyewear, which are referenced 1, comprises a frame 2 which, itself, comprises a frontal part 3, or face, in which two optical glasses 4 are embedded and which laterally extends to two rims 5, as a single piece with the face, and two arms 6 hinged to the opposite ends of the side rims 5.

The frame 2 is of a plastic material transparent to ionising radiation and the frontal protection is only ensured by the glasses 4 which are lead glasses.

In the example shown in FIG. 1A, the side protection is ensured by two small substantially rectangular lead plates 7, which are applied to a part of the side rims 5, at a distance from the outer edges of the lead glasses 4.

In the example shown in FIG. 1B, the side protection is ensured by two substantially triangular lead glass pieces 7 which are integrated into a part of the side rims 5, also at a distance of the outer edges of the lead glasses 4.

In both cases, the side protective elements typically have a lead equivalence of 0.35 mm.

FIGS. 2A and 2B are now referred to, which schematically and partially represent a person's head 8, in a left side view, wearing a radiation protective eyewear such as illustrated in FIG. 1A, as well as this person's crystalline lens 9.

In FIGS. 2A and 2B, which are for showing limits of the frontal protection which is ensured by this eyewear, the left lead glass 4 is represented, in a transverse cross-section view. However, the face 3 of the frame is not represented since it is made in a material transparent to ionising radiation and the left small plate 7 is also not represented for legibility reasons.

As is visible in FIG. 2A, the frontal protection is limited to “directs” radiation impinging on lead glasses and an example of which is embodied by the arrow f1.

Thus, the crystalline lens is protected:
neither from “direct” radiation impinging on the upper and lower parts of the face of the frame and two examples of which are embodied by the arrows f2,
nor from “scattered” radiation which arise from interaction of “direct” radiation with skin zones surrounding the eyewear and one example of which is embodied by the arrow f3.

As a result, the non-radiation protected angular sector in the median vertical plane of the crystalline lens 9 corresponds to the sector denoted as S1 in FIG. 2B.

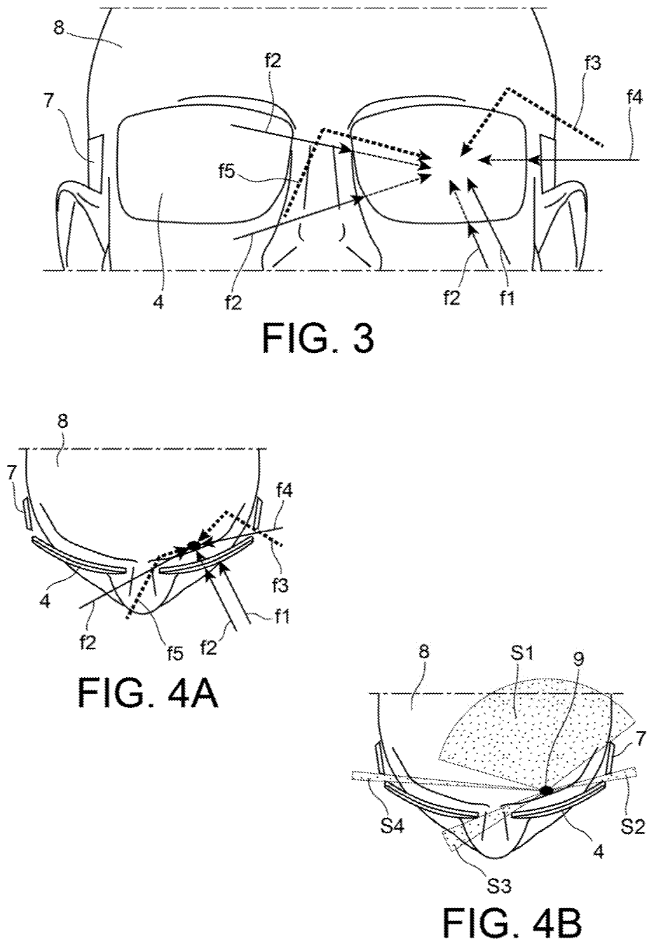

FIG. 3 is now referred to, which schematically and partially represents the head 8 shown in FIGS. 2A and 2B but in a front view.

In FIG. 3, which is for showing limits of frontal and side protections which are ensured by radiation protective eyewear such as illustrated in FIG. 1A, the lead glasses 4 and the small plates 7 of this eyewear are visible. However, the frame is not represented for the same reasons as above.

Here again, FIG. 3 shows that this eyewear protects the crystalline lens from “direct” radiation impinging on the lead glasses and an example of which is embodied by the arrow 11 but that it protects it:
neither from “direct” radiation impinging on the face of the frame and three examples of which are embodied by the arrows f2,
nor from “scattered” radiation which arise from interaction of “direct” radiation» with skin zones surrounding the eyewear and an example of which is embodied by the arrow f3.

It also shows that this eyewear does not either protect the crystalline lens from:
“direct” radiation impinging on parts of the side rims which are located between the side ends of the face of the frame and the lead small plates and an example of which is embodied by the arrow f4, and
“scattered” radiation which arise from interaction of direct radiation with skin zones such as the nose root and the upper part of dorsum of nose, which, although covered by the frame, are not protected by the same and an example of which is embodied by the arrow f5.

Another illustration of limits of frontal and side protections provided by radiation protective eyewear such as illustrated in FIG. 1A is given in FIGS. 4A and 4B which schematically and partially represent the head 8 shown in FIGS. 2A, 2B and 3 but in a top view.

FIG. 4A repeats different radiation types embodied by arrows f1 to f5 in FIG. 3 whereas FIG. 4B shows 4 angular non-radiation protected sectors in the median horizontal plane of the crystalline lens 9, respectively denoted as S2, S3, S4 and S5, with this eyewear.

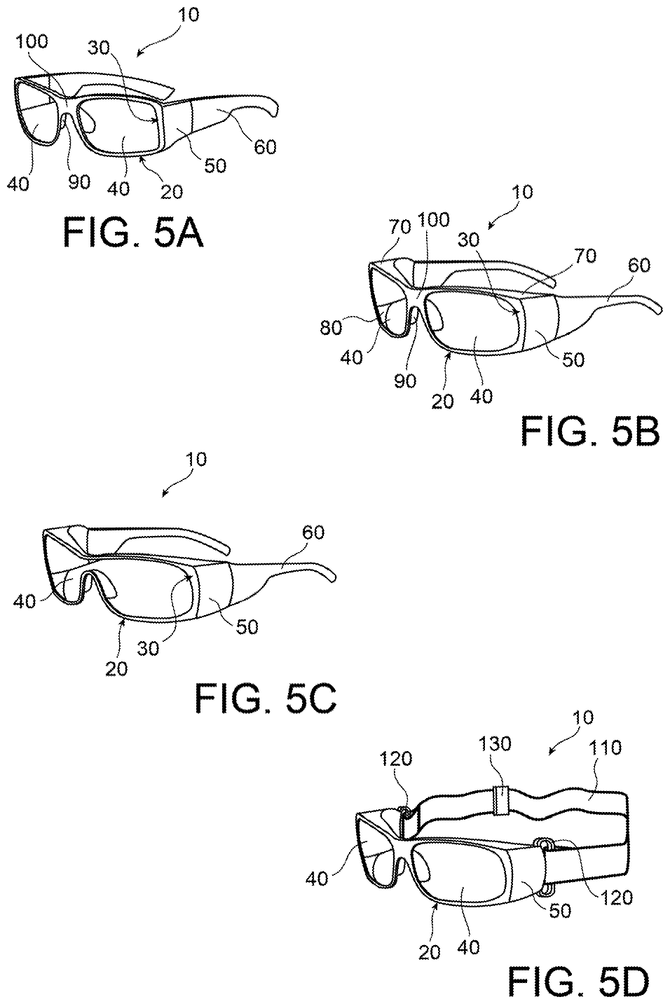

FIGS. 5A and 5B are now referred to, which illustrate two first examples of radiation protective eyewear of the invention.

This eyewear, which is referenced 10, comprises a frame 20 which, itself comprises a frontal part 30, or face, in which two optical glasses 40 are embodied and which laterally extends to two rims 50, as a single piece with the face, and two arms 60 hinged—via two hinges (not represented)—to the opposite ends of the side rims 50.

As in the eyewear illustrated in FIGS. 1A and 1B, the glasses 40 are lead glasses.

However, unlike the eyewear illustrated in FIGS. 1A and 1B, the face 30 of the frame as well as the side rims 50 are made of a radiation attenuating material which comprises a polymer matrix, for example of cellulose acetapropionate, polyamide or copolyamide, of epoxide resin or polyurethane, in which a radiation attenuating load is dispersed comprising, for example, particles of lead, lead oxide, erbium, erbium oxide, praseodymium, praseodymium oxide, a mixture of erbium oxide and praseodymium oxide, a mixture of erbium oxide, praseodymium and bismuth oxide, or a mixture of bismuth oxide, tungsten oxide and lanthanum oxide, or consisting of such particles.

In the example illustrated in FIG. 5A, the frame 20 is of the conventionally called “rectangular” type in the field of optical frames, that is with a substantially rectangular face 30 and having a low bending angle (lower than 10°) and side rims 50 which extend substantially perpendicular to the face 30. The height of these side rims is substantially equivalent to that of the face 30 at the junction of said side rims on said face.

In the example illustrated in FIG. 5B, the frame 20 is a more wrap-around frame than that of the eyewear illustrated in FIG. 5A, with a face 30 which has a stronger bending angle and the presence of two supra-orbital protective elements 70 and two infra-orbital protective element 80 which are as a single piece with the face 30 and side rims 50.

In the example illustrated in FIG. 5A as in the example illustrated in FIG. 5B, the frame 20 further comprises a saddle bridge 90, which can be assembled to the basis of the nose bridge 100 joining both optical glasses 40, in which case it is advantageously made of a flexible elastomer material such as a silicone, possibly containing a radiation attenuating load, or can be integrated into the nose bridge 100, that is as a single piece with this nose bridge.

FIG. 5C illustrates a third example of radiation protective eyewear 10 of the invention. This eyewear only differs from that shown in FIG. 5B in that the frame 20 is “mono-shield”, that is with a face 30 in which a single optical glass 40 is embodied, which extends from a side edge to the other of said face and which thus covers a user's both orbital areas in wearing condition.

FIG. 5D illustrates a fourth example of radiation protective eyewear 10 of the invention which only differs from that shown in FIG. 5B in that the frame 20 comprises a holding headband 110 instead of arms.

This holding headband, which is preferentially of an elastic material, for example an elastic textile or elastomer such as neoprene, is connected to the opposite ends of the side rims 50 of the frame 20, for example via two loops 120 which are as a single piece with these side rims and at which the ends of the headband form a closed loop by a fastening element (not represented) of the snap fastener, rivet, repositionable adhesive, hook and loop strip (or Velcro™) type or similar. The holding headband is furthermore provided with an element 130 for opening or closing it and/or adjusting the size thereof to the circumference of a user's head.

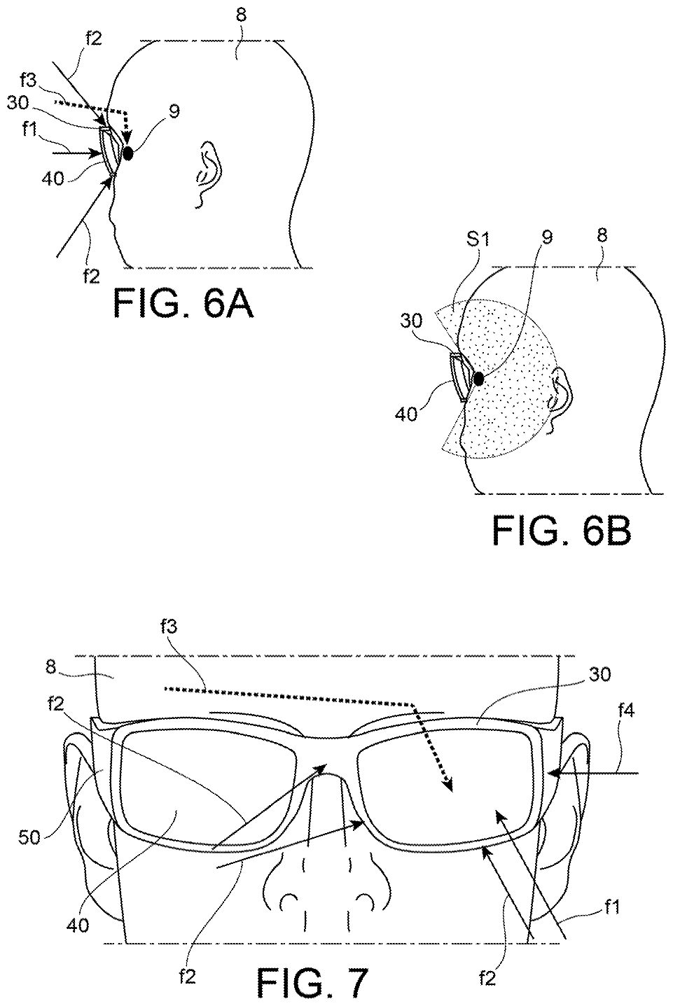

FIGS. 6A and 6B are now referred to, which are similar representations to those of FIGS. 2A and 2B but for a person wearing radiation protective eyewear such as illustrated in FIG. 5A. For this reason, in these figures, the face 30 of the frame 20, is represented in a transverse cross-section view.

As shown in FIG. 6A, the eyewear of the invention enables the crystalline lens 9 to be protected non only from “direct” radiation impinging on the lead glasses and an example of which is embodied by the arrow 11, but also from “direct” radiation impinging on the upper and lower parts of the face of the frame and two examples of which are embodied by the arrows f2.

For this reason, as is visible in FIG. 6B, the non-radiation protected angular sector in the median vertical plane of the crystalline lens 9, denoted as S1, obtained with the eyewear of the invention, is significantly reduced relative to that shown in FIG. 2B.

FIG. 7 is also a representation analogous to that of FIG. 3 but for a person wearing radiation protective eyewear such as illustrated in FIG. 5A.

This figure shows that this eyewear protects the crystalline lens from:
“direct” radiation impinging on the lead glasses and an example of which is embodied by the arrow 11,
“direct” radiation impinging on the face of the frame and three examples of which are embodied by the arrows f2, and as well
“direct” radiation impinging on the side rims and an example of which is embodied by the arrow f4.

Accordingly, “scattered” radiation arising from interaction of direct radiation with skin zones covered by the frame such as that embodied in FIG. 3 by the arrow f5, are also suppressed.

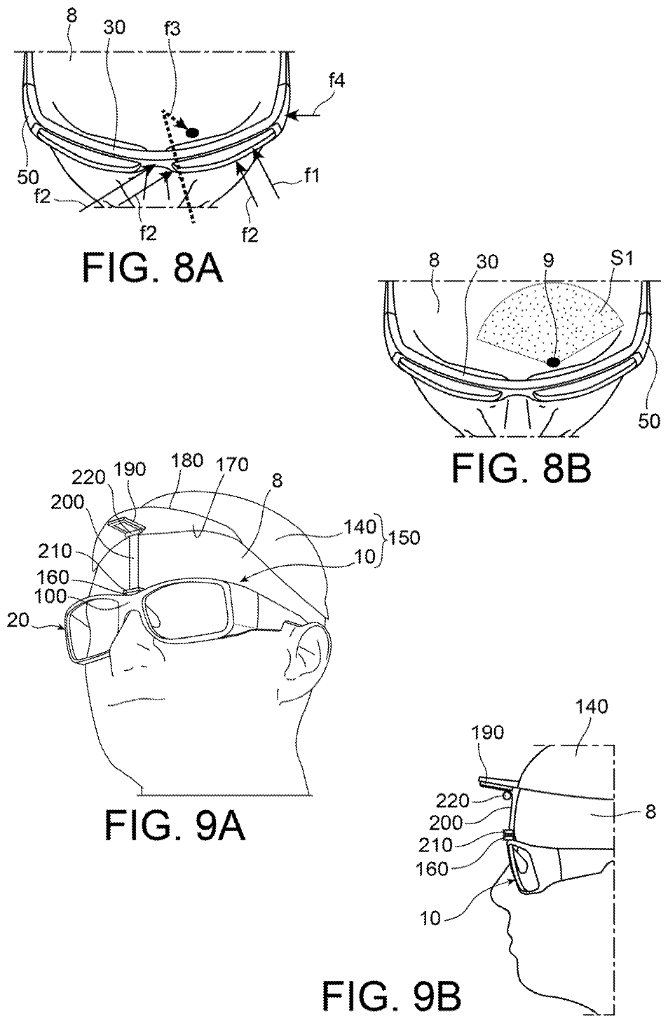

This is also shown by FIG. 8A which is a representation analogous to that of FIG. 4A but for a person wearing radiation protective eyewear such as illustrated in FIG. 5A and in which the different radiation types embodied by the arrows 11 to f5 in FIG. 7 are repeated.

For this reason, as is visible in FIG. 8B which is to be compared with FIG. 4B since it is a representation analogous to that of FIG. 4B but for a person wearing radiation protective eyewear such as illustrated in FIG. 5A, there remains, with the radioprotective eyewear of the invention, only one non-radiation protective angular sector in the median horizontal plane of the crystalline lens 9, that is sector S1, which is however significantly reduced relative to the angular sector S1 visible in FIG. 4B.

By way of example, radiation protective eyewear such as illustrated in FIG. 5A has been made with SCHOTT SF6 lead glasses, a frame and 5 mm-thick side rims, comprising bismuth sesquioxide Bi₂O₃ dispersed in a polyurethane matrix in an amount of 50% by mass to obtain a lead thickness of 0.50 mm. This eyewear weighs 92 g.

Therefore, in order not to lose the ergonomic characteristic, the invention suggests wearing this eyewear together with a weight take-up device 140 as illustrated in FIGS. 9A and 9B which schematically represent a person's head 8, in a front view (FIG. 9A) and a side view (FIG. 9B), respectively, wearing an example of a radiation protective eyewear 10/weight take-up device 140 assembly of the invention.

As is visible in these figures, in this assembly, which is referenced 150, the frame 20 of the eyewear, which is substantially of the same type as that illustrated in FIG. 5A, is fitted with a magnet 160 which is assembled to the upper edge of the nose bridge 100 but which could also be integrated into this nose bridge.

The weight take-up device 140 is itself in the form of a cap.

The lower part 170 of the peak 180 of this cap is fitted with a membrane 190 for holding a tongue 200 the lower end of which comprises a magnet 210 with a similar size to that included by the frame 20 of the eyewear but with an opposite polarity and the upper end of which is removably fastened to this member, for example by clipping, a repositionable adhesive or a Velcro™ type hook and loop strip. In the latter case, the tongue itself can be a hook and loop strip.

A cylindrical piece 220, integral with the holding member 190, enables the whole part of the tongue which is located between this piece and the magnet 210 to be held directly above the nose bridge 100 of the eyewear.

Removably fastening the upper end of the tongue 200 to the holding member 190 enables the length of the part of the tongue 200 which is included between the piece 220 and the magnet 210 to be adjusted to the height of a user forehead which is included between the peak 180 and the nose root of this user depending on wearing conditions of the cap by the latter.

Once this adjustment is made, the frame 20 of the eyewear is automatically secured to the cap by the magnetic effect which is established between the magnets 160 and 210 and part of the weight exerted by the frame 20 on the user's nose root is automatically taken up by this cap.

What is claimed is:

1. A frame for protective eyewear against ionising radiation, comprising a frontal part being laterally extended by two side protective elements, and a device for holding the frame on a user's face in wearing condition, wherein the side protective elements are as a single piece with the frontal part and the frontal part and the side protective elements are made of a radiation attenuating material comprising a polymer matrix in which a radiation attenuating load is dispersed.

2. The frame for eyewear of claim 1, wherein the radiation attenuating load comprises particles of at least one metal, the metal being lead, a rare earth, bismuth, antimony, tin, tungsten, barium, tantalum, or particles of an alloy or oxide thereof.

3. The frame for eyewear of claim 2, wherein the radiation attenuating load comprises particles of lead, lead oxide, erbium, erbium oxide, praseodymium, praseodymium oxide, a mixture of erbium oxide and praseodymium oxide, a mixture of erbium oxide, praseodymium oxide and bismuth oxide, or a mixture of bismuth oxide, tungsten oxide and lanthanum oxide.

4. The frame for eyewear of claim 1, wherein the radiation attenuating load comprises particles of bismuth or bismuth oxide.

5. The frame for eyewear of claim 1, wherein the polymer of the matrix is a cellulose acetate, a cellulose acetopropionate, a polyamide, a copolyamide, an epoxide resin or a polyurethane.

6. The frame for eyewear of claim 1, wherein the radiation attenuating material comprises from 20% to 95% by mass of the radiation attenuating load.

7. The frame for eyewear of claim 1, wherein the frontal part is configured to embed one or two optical glasses.

8. The frame for eyewear of claim 1, further comprising two supraorbital protective elements as a single piece with the frontal part and the side protective elements.

9. The frame for eyewear of claim 1, further comprising two infraorbital protective elements as a single piece with the frontal part and the side protective elements.

10. The frame for eyewear of claim 1, wherein the frame holding device comprises two arms hinged to the side protective elements or a headband connected to the side protective elements.

11. The frame for eyewear of claim 1, which further comprises a saddle bridge as a single piece with the frontal part or assembled to the frontal part.

12. Protective eyewear against ionising radiation, comprising one or two radiation attenuating optical glasses embedded in the frame of claim 1.

13. The eyewear of claim 12, wherein the radiation attenuating optical glass(es) is lead glass(es).

14. The Eyewear of claim 12, wherein the frame comprises a first attaching element which is configured to attach to a second attaching element comprised by a weight take-up device for eyewear when the eyewear and the weight take-up device are worn by a user, the eyewear being worn on the user's face and the weight take-up device being worn on the user's skull.

15. The eyewear of claim 14, wherein the first attaching element is a magnet assembled to the frame or integrated into the frame.

16. An assembly comprising the radiation protective eyewear of claim 12, and a weight take-up device, wherein the eyewear comprises a first attaching element, the weight take-up device comprises a second attaching element and wherein the first and second attaching elements are configured to attach to each other when the eyewear and the weight take-up device are worn by a user, the eyewear being worn on the user's face and the weight take-up device being worn on the user's skull.

17. The assembly of claim 16, wherein the first and second attaching elements are magnets with opposite polarity.

18. The assembly of claim 16, wherein the weight take-up device is configured to cover the top of the user's skull or to surround only the frontal part of the user's skull in wearing condition.
